(19) United States
(12) Patent Application Publication
(10) Pub. No.: US 2018/0344874 A1
(43) Pub. Date: Dec. 6, 2018

(54) Cysteine Peptide-Containing Health Drink

(71) Applicant: For Days Co., Ltd., Tokyo (JP)

(72) Inventors: Yoshiki Takahashi, Tokyo (JP); Koji Usumi, Tokyo (JP)

(21) Appl. No.: 16/034,949

(22) Filed: Jul. 13, 2018

Related U.S. Application Data
(63) Continuation of application No. 14/350,519, filed on Apr. 9, 2014.

(30) Foreign Application Priority Data
Oct. 12, 2011 (JP) 2011-225356

(51) Int. Cl. A61K 48/00; A61K 31/4415; A61K 8/27; A61K 8/60; A61K 8/65; A61K 8/67; A61K 8/73; A61Q 19/08; A61K 38/39; A23L 33/17; A23L 33/16; A23L 33/15; A23L 33/13; A23L 33/10; A61K 31/714; A23L 2/66; A61K 31/375; A23L 2/52; A61K 31/51; A61K 31/525; A61K 31/713; A61K 31/728; A61K 31/737; A61K 33/30; A61K 38/06; A61K 38/17

(52) U.S. Cl. CPC A61K 48/005; A23V 2002/00; A23V 2250/712; A23V 2200/00; A61K 8/27; A61K 8/606; A61K 8/65; A61K 8/673; A61K 8/675; A61K 8/676; A61K 8/735; A61Q 19/08; A61K 2800/92; A61K 38/39; A23L 33/17; A23L 33/16; A23L 33/15; A23L 33/13; A23L 33/10; A61K 31/714; A23L 2/66; A61K 31/375; A23L 2/52; A61K 31/51; A61K 31/525; A61K 31/713; A61K 31/728; A61K 31/737; A61K 33/30; A61K 38/063; A61K 38/1709; A23V 2200/318; A23V 2250/1642; A23V 2250/31; A23V 2250/5422; A23V 2250/704; A61K 31/4415

(57) ABSTRACT

An object is to provide a health drink which gives a real feeling of a preventive effect and ameliorative effect against skin aging and stress, and it is confirmed that when a drink in which glutathione is added to a conventional drink containing chondroitin, hyaluronic acid, and vitamins is taken, a concentration of blood DHEA-S, which is considered to be an indicator of rejuvenation, is significantly elevated, promoting gene expressions of olfactory receptors and the like, and remarkably ameliorating skin condition, total mood disturbance, autonomic nerve balance, and overall health condition.

### CROSS-REFERENCE TO RELATED APPLICATIONS

This patent application is a continuation application of U.S. patent application Ser. No. 14/350,519 filed on Apr. 9, 2014 and which claims priority to International Application No. PCT/JP2012/006525 filed Oct. 11, 2012 which claims the priority of JP 2011-225356 filed on Oct. 12, 2011. The contents of which is incorporated by reference in its entirety.

### TECHNICAL FIELD OF THE INVENTION

The present invention relates to a health drink comprising a water-soluble nucleoprotein, oligoribonucleotide (oligo RNA), zinc, collagen, chondroitin, hyaluronic acid, vitamins, and glutathione, and also a composition for elevating a concentration of blood dehydroepiandrosterone sulfate (DHEA-S), a composition for promoting an expression of olfactory receptor genes, and a composition for ameliorating or preventing aging or stress, all of which comprises containing a water-soluble nucleoprotein, oligo RNA, zinc, collagen, chondroitin, hyaluronic acid, vitamins, and glutathione.

With recent increasing health consciousness, and also for preventing diseases in the upcoming super-aging society, foods and drinks and components thereof have been under development to supplement specific nutrients often deficient in daily life, to achieve health maintaining and promoting effects, and further, with special attentions, to enhance the specific “biological regulatory functions” such as aging prevention, rejuvenation, and stress release. For example, health drinks containing water-soluble nucleoproteins, collagen, chondroitin, hyaluronic acid, vitamins, and the like, have been proposed (see for example, Patent Document 1) and marketed already by the present applicant.

For example, glutathione is an in vivo antioxidant present in each tissue of the majority of living creatures, and it is known that 2 kinds of reduced glutathione and oxidized glutathione are formed by the so-called redox cycle by glutathione peroxidase and glutathione reductase to maintain the function as the antioxidant (see for example, Patent Document 2).

It is reported that glutathione, utilizing such function of glutathione, can be used for stress preventing agents and stress ameliorating foods as the antioxidant (see for example, Patent Document 3 and Patent Document 4). Also, a wide variety of glutathione-containing foods and drinks have been proposed such as glutathione-containing sleep inducing drugs and stress insomnia ameliorating agents (see for example, Patent Document 5), drinks showing a glutathione content of 0.01 to 1.0 wt % (see for example, Patent Document 6), and glutathione-containing drinks showing an oxidized glutathione content of at least 50 wt % in the total glutathione content (see for example, Patent Document 7).

On the other hand, DHEA-S is known as one of the hormones, which have been drawing attention as an indicator of rejuvenation (see for example, Non-patent Document 1). DHEA-S is an intermediary metabolite in the male sex hormone synthesis and a sulfate conjugate of dehydroepiandrosterone (DHEA), which is also an intermediary metabolite. It is known that DHEA-S declines with ages in both male and female from young to old. DHEA-S has a longer blood concentration half-life than DHEA and no remarkable fluctuation is observed (see for example, Patent Document 8). For this reason, it is considered to be used as an objective indicator for indicating the degree of aging. Further, in patients under stress, it is confirmed that DHEA-S reduces (see for example, Non-patent Document 2).

Naturally, rejuvenating effects appear to be expected with an increased blood DHEA-S concentration, but it is reported that a DHEA administration experiment conducted on 12 male subjects, the average age 59, showed elevated levels of the endogenous DHEA-S but no clinical effects were acknowledged in any indicators for adipocyte, insulin, LDL cholesterol, HDL cholesterol, testosterone, estradiol, or the like (see for example, Non-patent Document 3). Further, at present, it is not recommended to administer DHEA to post menopausal women for the therapeutic purposes (see for example, Non-patent Document 4).

Granular foods with rejuvenating effects including Panax notoginseng as the main ingredient and further containing turmeric and Gloydius blomhoffii (see for example, Patent Document 9) are proposed in the form of foods or pharmaceutical preparations for the purpose of elevating a blood DHEA-S level. However, these ingredients are not readily available and very expensive. Further, for elevating the endogenous DHEA-S, an endogenous DHEA-S elevating preparation (see for example, Patent Document 8), which has as the main component autologous lymphocytes proliferated and activated by culturing lymphocytes collected from a subject in a culture broth containing solid phase anti-CD3 antibodies and interleukin-2, has been proposed, but the production method is cumbersome and cannot easily be used.

Meanwhile, it is known that the olfactory function declines as one gets older, but Axel and Buck identified the mechanism on complicated combinations of smell receptors which proceeded the analysis of expression patterns of olfactory receptors (OR). For example, it is reported that when 9 representative OR genes were detected by the in situ hybridization from mice as the subjects, each of these genes had different expression peaks but 6 of which had downregulated expressions with age (see for example, Non-patent Document 5).

### PRIOR ART DOCUMENTS

### Patent Documents

Patent Document 1: Japanese unexamined Patent Application Publication No. 2003-325149
Patent Document 2: Japanese unexamined Patent Application Publication No. 2006-348052
Patent Document 3: Japanese unexamined Patent Application Publication No. 2010-030901
Patent Document 4: Japanese unexamined Patent Application Publication No. 08-275752
Patent Document 5: Japanese unexamined Patent Application Publication No. 2008-056628
Patent Document 6: Japanese unexamined Patent Application Publication No. 06-078713
Patent Document 7: Japanese unexamined Patent Application Publication No. 05-146279
Patent Document 8: Japanese unexamined Patent Application Publication No. 2010-077106
Patent Document 9: Japanese unexamined Patent Application Publication No. 2005-204504

### Non-Patent Documents

Non-patent Document 1: Science 2002, Vol. 297 no 5582 p. 811
Non-patent Document 2: Nippon Ronen Igakkai Zasshi (in Japanese) (Japanese Journal of Geriatrics), Vol. 31, No. 2, 85-94
Non-patent Document 3: The Aging Male, 2003, Vol. 6, No. 3, pages 151-156
Non-patent Document 4: Menopause Int 2007; 13: 75-78
Non-patent Document 5: Chem Senses, Vol. 34, No. 8, 695-703, 2009

### Object to be Solved by the Invention

An object of the present invention is to provide a health drink and the like, which give a real feeling of a preventive effect and ameliorating effect against aging and stress.

### Means to Solve the Object

The present inventors, from viewpoint of reinforcing nutrients which act particularly in the nucleus, continued studying on novel additive components to develop a drink with objectively assessable efficacies superior to those of conventional health drinks which claim various effects, and found that when subjects take an improved drink in which a glutathione-containing yeast extract is newly added to the conventional health drinks containing water-soluble nucleoproteins, collagen, chondroitin, hyaluronic acid, vitamins, and the like, the subjects benefit additional effects from the effects of the conventional health drinks and the effects of glutathione, and further synergistic ameliorative effects on skin condition, total mood disturbance, autonomic nerve balance, and overall health condition. Biological evidence for supporting such synergistic effects has been continuously studied, and surprisingly it was confirmed that when taking the above improved drink, the subjects had a significantly elevated concentration of blood DHEA-S, which is considered to be an indicator of rejuvenation, together with a reduced LDL cholesterol, and also, at the gene level, the expressions of olfactory receptor genes and the like are promoted. The present invention has been accomplished based on these findings.

More specifically, the present invention relates to [1] a health drink comprising a water-soluble nucleoprotein, oligo RNA, zinc, collagen, chondroitin, hyaluronic acid, a vitamin, and glutathione; [2] the health drink according to [1], wherein the vitamin includes one or more B vitamins selected from the group consisting of vitamin B1, vitamin B2, vitamin B6, and vitamin B12, and vitamin C; [3] the health drink according to [1] or [2], wherein a glutathione-containing yeast extract is used as the glutathione; [4] the health drink according to [3], wherein the glutathione-containing yeast extract contains more oxidized glutathione than reduced glutathione; [5] the health drink according to any one of [1] to [4], wherein 6 to 60 mg of glutathione per 1000 mL is added.

Further, the present invention relates to [6] a composition for elevating a concentration of blood DHEA-S, the composition comprising a water-soluble nucleoprotein, oligo RNA, zinc, collagen, chondroitin, hyaluronic acid, a vitamin, and glutathione; [7] a composition for promoting an expression of an olfactory receptor gene, the composition comprising a water-soluble nucleoprotein, oligo RNA, zinc, collagen, chondroitin, hyaluronic acid, a vitamin, and glutathione; [8] a composition for suppressing an expression of TNFRSF10C gene or TNFSF14 gene, the composition comprising a water-soluble nucleoprotein, oligo RNA, zinc, collagen, chondroitin, hyaluronic acid, a vitamin, and glutathione; [9] a composition for ameliorating or preventing aging or stress, the composition comprising a water-soluble nucleoprotein, oligo RNA, zinc, collagen, chondroitin, hyaluronic acid, a vitamin, and glutathione; [10] the composition according to any one of [6] to [9], wherein the vitamin includes one or more B vitamins selected from the group consisting of vitamin B1, vitamin B2, vitamin B6, and vitamin B12, and vitamin C; [11] the composition according to any one of [6] to [10], wherein a glutathione-containing yeast extract is used as the glutathione; [12] the composition according to [11], wherein the glutathione-containing yeast extract contains more oxidized glutathione than reduced glutathione.

Examples of other embodiments of the present invention include a method for elevating a blood DHEA-S concentration and/or a method for promoting an expression of olfactory receptor genes comprising orally administering to a subject a composition containing a water-soluble nucleoprotein, oligo RNA, zinc, collagen, chondroitin, hyaluronic acid, a vitamin, and glutathione; and use of a composition containing a water-soluble nucleoprotein, oligo RNA, zinc, collagen, chondroitin, hyaluronic acid, a vitamin, and glutathione for preparing an agent for elevating a blood DHEA-S concentration and/or an agent for promoting an expression of olfactory receptor genes.

### Effect of the Invention

When orally taking the drink or composition of the present invention, a subject can have a real feeling of the effects on enhanced skin condition, improved autonomic nerve balance, improved total mood disturbance, enhanced health condition, and the like, whereby aging prevention or improvement and stress prevention or improvement are achieved together with the effects on elevating a blood DHEA-S concentration, reducing LDL cholesterol, and/or promoting expressions of olfactory receptor genes and further growth hormone genes, and the like.

### BRIEF DESCRIPTION OF THE DRAWINGS

FIG. 1 Images showing embodiments of the facial skin condition of a subject from Group B when analyzed by “VISTA” on items of stains, pores, pigmentation irregularities, and ultraviolet ray stains. (a) shows the facial skin condition of a subject before taking drink b, (b) shows the facial skin condition of the same subject after taking drink b for 5 weeks.

FIG. 2 Shown is the correlation, revealed by “VISTA”, between reduction (improvement) of pores and reduction (improvement) of pigmentation irregularities in terms of the facial skin improvement in the subjects from each of Groups A, B, and C before and after taking any one of drinks a, b, and c for 5 weeks.

FIG. 3 Shown is the improvement rate of the average value in the subjects from each of Groups A, B, and C in terms of the item of autonomic nerve balance by “Kiritsu meijin (in Japanese) (Stand-up expert)” before and after taking any one of drinks a, b, and c for 5 weeks.

FIG. 4 Shown are the fluctuations in blood DHEA-S concentrations of the subjects from each of Groups A, B, and C before and after taking any one of drinks a, b, and c for 5 weeks. The vertical axis shows the blood DHEA-S concentration (μg/dL).

FIG. 5 Shown are the average elevation rates (%) of blood DHEA-S concentrations of the subjects from each of Groups A, B, and C after, as opposed to before, taking each drink.

FIG. 6 With the probes showing an expression variation of significantly 1.2 times or more in each subject from Group A, Group B, and Group C before and after taking any one of drinks a, b, and c, how the probe expression variations in each group overlap are represented in the form of Venn diagrams for the expression increase and expression decrease, respectively. (a) investigates the probes showing a 1.2 times or more increase, and (b) investigates the probes showing a 1.2 times or more decrease.

FIG. 7 Of the probes varying specifically to Group B, the genes belonging to the “Receptor activity” under the molecular function of Gene Ontology are excerpted and listed.

FIG. 8 Of the probes varying specifically to Group B, the genes belonging to the “Receptor binding” under the molecular function of Gene Ontology are excerpted and listed.

FIG. 9 Shown are the results of comparative analysis on skin firmness tests with identical twins who took the drinks in different patterns. (a) Shown is the graph of the first twin sister who took drink a, and subsequently took drink b with a washout period therebetween. (b) Shown is the graph of the second twin sister who took drink b, and subsequently took drink a with a washout period therebetween.

FIG. 10 Shown are the increase and reduction of creatinine during the test period of the identical twins who took the drinks in different patterns.

FIG. 11 Shown are the increase and reduction of total cholesterol during the test period of the identical twins who took the drinks in different patterns.

FIG. 12 Shown are the increase and reduction of LDL cholesterol during the test period of the identical twins who took the drinks in different patterns.

FIG. 13 The increase and decrease of numerical values obtained using AMSAT with the identical twins before and after taking drink a.

FIG. 14 The increase and decrease of numerical values obtained using AMSAT with the identical twins before and after taking drink b.

FIG. 15 With [FTS] and [STS] of the twins and each subject from Group B, the numbers of probes which showed an expression increase of significantly 1.2 times or more before and after taking drink b are represented in the form of Venn diagrams.

### DETAILED DESCRIPTION OF THE INVENTION

The health drink of the present invention is not particularly limited as long as it contains a water-soluble nucleoprotein, oligo RNA, zinc, collagen, chondroitin, hyaluronic acid, a vitamin, and glutathione, and further the method for manufacturing such a drink is not also particularly limited. Each component can be added and mixed separately, or all of the components can be added and mixed simultaneously, or mixtures of 2 or more components are separately mixed to manufacture the drink.

The above health drinks are not clearly scientifically or legally defined but contain components which are acknowledged to improve the physical predisposition and enhance the health condition, and referred to as the collective term which is distinguished from other drinks with the social recognition.

Generally, glutathione is roughly classified into reduced glutathione (GSH) and oxidized glutathione (GSSG). GSH is a tripeptide with the structure of 5-L-glutamyl-L-cysteinylglycine, and GSSG is a compound in which 2 GSH molecules are bonded by an S—S bond. Both of them are biosynthesized in vivo, but since GSSG is converted to GSH by glutathione reductase in vivo, the same action as GSH is considered to be provided even when GSSG is administered.

Glutathione according to the present invention can be either type as long as it can be used as food, and examples include glutathiones derived from marine microalgae such as Crypthecodinium cohnii (see for example, Japanese unexamined Patent Application Publication No. 9-292), glutathiones derived from yeasts belonging to peroxide resistant genus Saccharomyces, genus Hansenula, genus Endomycopsis, genus Saccharomicodes, genus Nematospora, genus Candida, genus Torulopsis, genus Brettanomyces, and genus Rhodotorula, glutathiones derived from microorganisms belonging to genus Escherichia, genus Alcaligenes, or genus Proteus (see for example, Japanese unexamined Patent Application Publication No. 8-70884).

Specific examples of the glutathione-containing yeast include yeasts belonging to genus Saccharomyces such as Saccharomyces cerevisiae, Saccharomyces rouxii, and Saccharomyces carlsbergensis, and yeasts belonging to genus Candida such as Candida utilis (Torula yeast), Candida tropicalis, Candida lipolytica, and Candida flaveri.

Further, examples of the method for producing the glutathione-containing yeast containing a high content of glutathione (high glutathione content yeast) include a method of obtaining in the form of cultured yeast by a method in which 3 kinds of amino acids are added (see for example, Japanese unexamined Patent Application Publication No. 53-94089), a method in which methionine and glutamic acid are added to medium during the yeast culture to increase an SAM content and glutathione content in the yeast (see for example, Japanese unexamined Patent Application Publication No. 2009-017849), or the like, and also a method in which a yeast mutant strain such as a yeast mutant strain having at least a part of the DOA1 gene and MET30 gene deleted or mutated by mutation treatment (see for example, Japanese unexamined Patent Application Publication No. 2010-029147), or a yeast mutant strain belonging to genus Candida, growable in the presence of a polyene antibiotic and capable of containing a large amount of GSSG (see Japanese unexamined Patent Application Publication No. 2003-284547). When a yeast mutant strain is used, it is preferred to use a high GSSG content mutated yeast wherein the amount of GSSG production is increased in the light of higher stability of GSSG in an aqueous solution than GSH. Preferable examples of such a high GSSG content yeast mutant strain include yeast mutant strains belonging to genus Candida, growable in the presence of a polyene antibiotic and capable of containing a large amount of GSSG, and specifically Candida utilis 1453B5 (FERM P-18789), Candida utilis 1483A6 (FERM P-18790), and mutants thereof (see Japanese unexamined Patent Application Publication No. 2003-284547) are preferred examples.

It is preferred for a microorganism such as the above glutathione-containing yeasts to be added in the form of extract of the microorganism since such a form can easily be added to foods and drinks, and examples of such a glutathione-containing yeast extract include those containing 2 mass % or more, preferably 8 mass % or more, more preferably 10 mass % or more, further preferably 11 mass % or more, and particularly 12 mass % or more, of glutathione.

Examples of the method for producing microorganism extracts such a glutathione-containing yeast include known methods such as those wherein a glutathione-containing yeast or the like is autolized, enzymatically treated, or heat extracted (extraction by a solvent such as hot water) and subsequently water-soluble components are separated, and a method for producing a yeast extract containing 10 wt % or more of oxidized glutathione belonging to genus Candida, growable in the presence of a polyene antibiotic and capable of containing a large amount of oxidized glutathione (see for example, Japanese unexamined Patent Application Publication No. 2004-283125) is the example. Alternatively, commercial products can be used as the glutathione-containing yeast extract, and examples of such a commercial product include Springer Hyp A (manufactured by Bio Springer SA) and Gluta-yeast extract N (manufactured by KYOWA HAKKO BIO CO., LTD.).

The above oligo RNA is not particularly limited as long as it is a low molecular RNA obtained by reducing the molecular weight of ribonucleic acid (RNA), but a specifically preferable example is the RNA which is extracted and purified from known yeasts such as brewer's yeast, torula yeast, milk yeast, and baker's yeast and reduced to a molecular weight to the extent that it contains 20 to 50% of fractions with a molecular weight of 1000 to 3000 and a larger amount, for example, 30 to 50%, of fractions with a molecular weight of 1000 or less than the amount of fractions with a molecular weight of 1000 to 3000.

Examples of the method for producing and obtaining the above oligo RNA include known methods such as a method wherein the RNA isolated, extracted, and purified from the above yeast is zymolyzed or hydrolyzed to reduce the molecular weight thereof, a method wherein the RNA is chemically or enzymatically synthesized, a method of purchasing a commercial product, but specifically a method wherein the RNA is prepared by hydrolyzing 3′,5′-phosphodiester bond thereof using a heat stable nuclease (see for example, Japanese unexamined Patent Application Publication No. 2007-23024) is a preferable example.

Also, the high glutathione content yeast described above can be used as an oligo RNA source of the above. When a yeast showing a high glutathione content is used, a glutathione-containing oligo RNA containing oligo RNA and 1% or more of glutathione (such is also sometimes called MATERNAL® RNA) is provided. When such a MATERNAL RNA is used, it can be treated, even without adding the above glutathione, as equal to the case wherein a suitable amount of glutathione is added.

The above water-soluble nucleoprotein is not particularly limited as long as it is a water-soluble nucleoprotein whose molecular weight is reduced by enzymatically treating the nucleoprotein contained in the nucleus of a biological cell, but a preferable example is a water-soluble nucleoprotein obtained by treating the nucleoprotein from soft roe of fish with nuclease and protease and containing 30% or more of oligonucleotide/nucleoside and oligopeptide showing a reduced molecular weight of 1000 to 3000 in the respect of confirmed effect for effectively suppressing oxidative damages of genes when impacts are applied such as exposure to ultraviolet ray irradiation and chemical substances (see for example, U.S. Pat. No. 3,978,716). An example of such a water-soluble nucleoprotein is Super Nuclegen.

The above hyaluronic acid encompasses a hyaluronic acid, which is a kind of proteoglycan and has a basic structure of linked units of disaccharide wherein the 1st position of β-D-glucuronic acid and the 3rd position of β-D-N-acetyl-glucosamine are bonded, derivatives thereof or salts thereof, which referred to as hyaluronic acids, and low molecular hyaluronic acid or hyaluronic acid decomposition products obtained by treating such hyaluronic acids with an enzyme such as hyaluronidase or by subjecting such hyaluronic acids to heating and pressurizing treatment. Examples of the above hyaluronic acid derivative include acetylated hyaluronic acid (see Japanese unexamined Patent Application Publication No. 8-53501), sulfated hyaluronic acid (see Japanese unexamined Patent Application Publication No. 10-195107), hyaluronic acid substituted with a bioorganic acid such as lactic acid (see Japanese unexamined Patent Application Publication No. 6-16702), and crosslinked hyaluronic acid (see Japanese unexamined Patent Application Publication No. 7-97401); examples of the hyaluronic acid and the salt of derivatives thereof include metal salts such as sodium salt, potassium salt, lithium salt, magnesium salt, and calcium salt, basic amino acid salts such as lysine salt, arginine salt, and histidine salt, ammonium salt, triethanolamine salt, and diisopropanolamine salt.

Examples of the method for producing and obtaining the hyaluronic acids include known methods such as a method wherein hyaluronic acids are isolated and extracted from the cockscomb of a chicken, the umbilical cord of a mammal, a metabolite of fishes or bacteria belonging to genus Lactobacillus or Streptococcus; a method wherein hyaluronic acids are chemically or enzymatically synthesized; and a method wherein a commercial product is purchased. Since a solution containing a hyaluronic acid usually has a high viscosity, a variety of commercial products such as cockscomb extracts and umbilical cord extracts containing decomposed and/or purified hyaluronic acid are sold to meet the type of products to which the product is added and the viscosity required. Examples include hyaluronic acid HA-F (manufactured by Kewpie Corporation), sodium biohyaluronate (manufactured by Shiseido Co., Ltd.), and hyaluronic acid FCH (manufactured by Kibun Food Chemifa Co., Ltd.), with hyaluronic acid HA-F (manufactured by Kewpie Corporation) being preferable.

The chondroitin of the present invention encompasses a chondroitin, which is a kind of glycosaminoglycan (mucopolysaccharide) and has a basic structure wherein sulfuric acid is linked to a polysaccharide chain containing two alternating monosaccharides, D-glucuronic acid (GlcA) and N-acetyl-D-galactosamine (GalNAc), derivatives thereof or salts thereof, which referred to as chondroitins. Examples of the chondroitin include chondroitin-4-sulfate (chondroitin sulfate A), dermatan sulfate (chondroitin sulfate B), chondroitin-6-sulfate (chondroitin sulfate C), chondroitin sulfate D, and chondroitin-4,6-sulfate (chondroitin sulfate E); examples of the chondroitin derivative include condensates of chondroitin and a reducing sugar such as glucose, galactose, maltose, or lactose, derivatives such as aryl ester, phosphoester, and sulfate ester; examples of the salt of chondroitin and derivatives thereof include alkali metal salts such as sodium salt and potassium salt, inorganic salts such as hydrochloride, sulphate, and ammonium salt, organic salts such as lactate, acetate, and triethanolamine salt.

Examples of the method for producing and obtaining the chondroitins include known methods such as a method wherein chondroitin is isolated and extracted from chondroitin-containing natural products such as a shark cartilage, shark fin extract; a method wherein chondroitin is chemically or enzymatically synthesized; and a method wherein a commercial product is purchased. Preferable examples of the commercial product include chondroitin-containing mucopolysaccharide protein complexes such as “shark cartilage extract (70)” (manufactured by NAKAHARA CO., LTD.).

The collagen of the present invention encompasses fibrous proteins with an structure of alternating units of a peptide fragment composed of “-glycine-amino acid-amino acid-” and constituting an extracellular matrix in tissues such as skin tissue, cartilage tissue, bone tissue, blood vessel tissue, organs, or tendons; and hydrolyzate thereof (collagen peptide), as well as derivatives thereof. Examples of the collagen derivative include atelo products, acylated products, and succinylated products of collagen and hydrolyzate thereof. Since the typical collagen has a molecular weight of about several ten thousand to 300000 and is usually water-insoluble, it is common to use a water-soluble collagen hydrolyzate (collagen peptide).

Examples of the usable method for producing and obtaining a collagen hydrolyzate include known methods such as a method wherein the dermis of an animal such as pig or cow is washed and bone is crushed, subsequently pretreatments required in accordance with collagen-containing tissues such as alkali treatment, neutralization, degreasing, or mineral removal are carried out to obtain an extract, which is then decomposed by an enzyme or the like, filtered, sterilized, spray-dried and powdered; and alternatively a commercial product can also be used. Examples of the commercial product include “S⋅ONE⋅S” (manufactured by PS CORPORATION), various collagen peptides derived from fishes (manufactured by RABJ CO., LTD.), “Gelita Sol LDA” (manufactured by Gelita AG), and “Solugel 5,000” (manufactured by PB Gelatin).

The zinc of the present invention is not particularly limited as long as it is in the form which can be added to food, and can be administered in the form such as zinc gluconate, zinc sulfate, or edible zinc yeast but it is preferred to be added in the form of an edible zinc-containing yeast since a higher absorption rate in vivo is acknowledged, and it is desirable to use a yeast containing 2 mass %, preferably 3 mass %, more preferably 4 mass % or more of zinc. Examples of the commercial product include edible yeasts (containing zinc) such as zinc yeast (manufactured by GROW), mineral yeast-Zn (manufactured by Oriental Yeast Co., Ltd.), and zinc yeast (manufactured by Bio Springer Corporate), with zinc yeast (manufactured by GROW) being preferable among them.

The vitamins contained in the health drink of the present invention are not particularly limited as long as they are vitamins, derivatives thereof, or salts thereof, which can render the above effects of the present invention. Examples include vitamin C (ascorbic acid), vitamin B1 (thiamine), vitamin B2 (riboflavin), vitamin B6 (pyridoxine), and vitamin B12 (cobalamin); examples of the vitamin C derivative or salt thereof include calcium ascorbate and sodium ascorbate; examples of the vitamin B1 derivative or salt thereof include thiamin hydrochloride, thiamine nitrate, bisthiamine nitrate, thiamine dicetylsulfate, fursultiamine hydrochloride, octotiamine, and benfotiamine; examples of the vitamin B2 derivative or salt thereof include riboflavin tetrabutyrate and riboflavin sodium phosphate; examples of the vitamin B6 derivative or salt thereof include pyridoxine hydrochloride, pyridoxalisol (pyridoxal phosphate), and pyridoxamine; examples of the vitamin B12 or derivative thereof or salt thereof include cyanocobalamin, hydroxocobalamin, hydroxocobalamin acetate, hydroxocobalamin hydrochloride, methylcobalamin, and adenosylcobalamin. Preferably 2 or more, more preferably 3 or more, and further preferably 4 or more, of the above vitamins are contained. Examples of the specific combination include vitamin C and vitamin B1, vitamin C and vitamin B2, vitamin C and vitamin B6, vitamin C and vitamin B12, vitamin C and vitamins B1 and B2, vitamin C and vitamins B1 and B6, vitamin C and vitamins B1 and B12, vitamin C and vitamins B2 and B6, vitamin C and vitamins B2 and B12, vitamin C and vitamins B6 and B12, vitamin C and vitamins B1, B2, and B6, vitamin C and vitamins B1, B6, and B12, vitamin C and vitamins B1, B6, and B12, vitamin C and vitamins B2, B6, and B12, as well as vitamin C and vitamins B1, B2, B6, and B12.

The amount of above each component added to the health drink of the present invention is not particularly limited but, for example, 6 to 60 mg of glutathione (0.05 to 0.5 g of a glutathione-containing microorganism extract), 0.1 to 5 g of oligo RNA, 0.5 to 10 g of a water-soluble nucleoprotein, 0.1 to 3 g of zinc yeast, 50 to 150 g of collagen, 0.01 to 0.5 g of chondroitin, 0.01 to 0.5 g of hyaluronic acid, and 5 to 20 g of vitamin C; preferably 12 to 42 mg of glutathione (0.1 to 0.35 g of a glutathione-containing microorganism extract), 0.5 to 2.0 g of oligo RNA, 2 to 8 g of a water-soluble nucleoprotein, 0.5 to 1.5 g of zinc yeast, 70 to 120 g of collagen, 0.05 to 0.3 g of chondroitin, 0.02 to 0.1 g of hyaluronic acid, and 9 to 15 g of vitamin C; and more preferably 18 to 36 mg of glutathione (0.15 to 0.30 g of a glutathione-containing microorganism extract), 1 to 1.5 g of oligo RNA, 4 to 6 g of a water-soluble nucleoprotein, 0.9 to 1.1 g of zinc yeast, 90 to 100 g of collagen, 0.08 to 0.25 g of chondroitin, 0.04 to 0.07 g of hyaluronic acid, and 11 to 13 g of vitamin C; are added per 1000 mL of the health drink of the present invention.

It is preferred that the drink of the present invention with the above formulation be taken 3 times a day, after breakfast, lunch, and dinner, 20 mL each, for 5 weeks, since the effects of the present invention are remarkably felt, but even when the amount and frequency of intake are increased or reduced as needed, the same effects can be achieved by taking it for an extended period of time. Also, the amount of intake of the health drink of the present invention can be determined by a pharmacologist or clinician of the relevant technical field using a known method, or alternatively can be determined subjectively based on the judgment of a drinker him/herself. For example, the amount and frequency of intake of the health drink of the present invention can also be increased or reduced as needed while the condition of a drinker is monitored, such as until a blood DHEA-S concentration of the drinker is elevated, until a total cholesterol value or LDL cholesterol value is reduced, or until a degree of stress perceived is lowered.

The drink of the present invention can contain, in consideration of the preference of consumers and to enhance the taste of the drink, sugars, sugar alcohols, and sweeteners. Examples of the sugar(s) include sucrose, fructose, glucose, lactose, and high fructose corn syrup; examples of the sugar alcohol(s) include xylitol, sorbitol, maltitol, erythritol, and mannitol; examples of the sweetener(s) include acesulfame potassium, sucralose, neotame, saccharin, aspartame, and stevia; and one or more of the sugars, sugar alcohols, and sweeteners can be added, respectively. Further, flavors can be added as needed to mask the smell of products made of animal ingredients, or to enhance the flavor of the drink. Furthermore, foods such as honey can also be added.

The drink of the present invention can further contain preservatives and emulsifiers as needed in the light of easiness to swallow and storability. Examples of the preservative include sodium benzoate and sorbic acid, with sodium benzoate being preferable; and examples of the emulsifier include glycerin fatty acid ester emulsifiers and sucrose fatty acid ester emulsifiers; and one or more of the preservatives and emulsifiers can be added, respectively.

The drink of the present invention can furthermore contain a gelatinizer to provide the easiness to swallow and to make the administration easier for a drinker with a poor ability to swallow. Examples of the gelatinizer include known gelatinizers such as gelatin, pectin, agar, carrageenan, gellan gum, glucomannan, and locust bean gum; and one or more of these gelatinizers can be added.

The composition of the present invention encompasses a composition for elevating a blood DHEA-S concentration, a composition for promoting an expression of olfactory receptor genes, and a composition for ameliorating or preventing aging or stress, all of which contain a water-soluble nucleoprotein, oligo RNA, zinc, collagen, chondroitin, hyaluronic acid, vitamins, and a glutathione-containing yeast. Also, the composition can be used as an ameliorating agent and/or preventive agent against aging or stress which, unlike pharmaceutical products, is free, or extremely low, of adverse effects after taken. Further, the composition of the present invention can be used in foods and drinks or materials thereof as a drink food additive for ameliorating and/or preventing aging or stress capable of imparting effects for elevating a blood DHEA-S concentration, promoting an expression of olfactory receptor genes, and further promoting an expression of growth hormone genes. The preparation form for the above aging or stress ameliorating and/or preventing agent, and drink food additive for ameliorating and/or preventing stress is not particularly limited, and solid preparations and liquid preparations can be formulated using a suitable carrier. For using the composition of the present invention in foods and drinks, the effective amount can be added or mixed at the stage of the production ingredient and/or at the stage of the completed product of foods and drinks.

Examples of the effect provided when the drink or composition of the present invention is taken include ameliorative or preventive effects on aging, and ameliorative or preventive effects on stress; examples of the ameliorative or preventive effect on aging include the improvement of skin aging, elevation of a blood DHEA-S concentration, reduction of blood LDL cholesterol, and expression promotion of olfactory receptor genes and/or expression promotion of growth hormones; examples of the ameliorative or preventive effects on stress include subjective effects such as a mood felt from enough sleep, and also objective effects such as ameliorative effects on total mood disturbance, autonomic nerve balance and function, and/or overall health condition, which are all measurable using a measuring apparatus.

The above skin aging is not particularly limited as long as it is the aging of skin even if it is physiological aging caused by the age, or photoaging caused by receiving an ultraviolet ray irradiation, and specific examples include one or more of observable pores, stains, pigmentation irregularities, and ultraviolet ray stains. An example of the effect for preventing and ameliorating the skin aging is the reduction of observable pores, reduction of stains, reduction of pigmentation irregularities, reduction of ultraviolet ray stains, and enhancement of skin condition in the overall assessment, after a subject took the drink or composition for a predetermined period of time. An example of the method for confirming such an effect is an analysis method using a VISIA Complexion Analysis (hereinafter also referred to as “VISIA”) (manufactured by Canfield), which is a skin image analysis counseling system considered excellent in the aspects of photographing a subject in a short time, setting a site to be analyzed liberally, and analyzing the same site from the second time on by adjusting analysis errors caused by deviations in photographed positions and color tone differences in photographs between before and after. Additionally, in each measurement item, when numerical values represented by the point are decreased, and/or numerical value of the overall assessment is increased, the aging of skin can be assessed as prevented or improved. Also, the effect can be assessed by carrying out a skin firmness test using a skin grip meter AS-GP1 (manufactured by Asahi Biomed).

An example of the ameliorative effect on the above total mood disturbance is the uplifting mood when the continuous mood of a subject is analyzed immediately after taking the drink or composition for a predetermined period of time. An example of the method for confirming such an effect is an analysis method which employs “Profile of Mood States: POMS”. POMS is an assessment test developed by McNair et al. (1971) and measures, based on the answers to 65 questions, temporary feeling or mood states consisting of 6 factors, more specifically, Tension-Anxiety: T-A, Depression-Dejection: D, Anger-Hostility: A-H, Vigor: V, Fatigue: F, and Confusion: C. Additionally, with the comparison of numerical values before and after a subject started taking the drink of composition, the “total mood disturbance” can be assessed as improved with significantly decreased numerical values for “Tension-Anxiety: T-A”, “Depression-Dejection: D”, “Anger-Hostility: A-H”, “Fatigue: F”, and “Confusion: C”, and with significantly increased numerical value for “Vigor: V”.

An example of the ameliorative effect on the above autonomic nerve balance and function is the improved autonomic nerve balance and function of a subject after taking the drink or composition for a predetermined period of time. An example of the method for confirming such an effect is an analysis method which employs “Kiritsu meijin (in Japanese) (Stand-up expert)” (manufactured by CROSSWELL CO., LTD.), an autonomic reflex real-time monitoring software that can diagnose the autonomic nerve function by the stress imposed when a posture is changed by standing up. When the autonomic nerve cannot respond well to the stress imposed when standing up, the orthostatic hypotension is sometimes caused, whereas when a mentally and physically healthy person stands up, the sympathetic nerve is customarily activated, peripheral blood vessels are contracted, and the heart rate is increased to maintain the blood circulation in the brain. Accordingly, the above method diagnoses the autonomic nerve balance at rest in a sitting position and also measures an instant heart rate when stress is imposed from a spontaneous stand-up to analyze frequency domain and time domain, and assesses the sympathetic nerve and parasympathetic nerve by the combination of such an analysis, thereby assessing the autonomic nerve function.

An example of the ameliorative effect on the above overall health condition is an enhanced overall health condition of a subject after taking the drink for a predetermined period of time. An examples of the method for confirming such an effect is an analysis method which uses AMSAT-HC (Auto Mastic System for Analysis Therapy—Holistic Concept), a device which can readily measure in a short time and is suitable for understanding the health condition of a subject and confirming therapy effects, and enables high reproducibility and accurate measurement. More specifically, 22 patterns of current value from six electrodes placed on the forehead, both hands, and both feet are measured, and the measured values are compared with the data bank information stored in the system and calculated by algorithm to provide data. It is understood that the data on 67 sites of the entire body can be obtained by a special algorithm including the waveform analysis, with a degree of deviation from the measurement result desired values indicated as the maximum of ±100%. Additionally, when the degree of deviation from the measurement result desired values gets smaller, the overall health condition can be assessed as improved.

An example of the effect in the above blood DHEA-S concentration is an elevated blood DHEA-S concentration of a subject after taking the drink for a predetermined period of time. Examples of the method for measuring a blood DHEA-S concentration include the radioimmunoassay method (RIA) and the tube solid phase method. Additionally, when a blood DHEA-S concentration of a subject is significantly elevated compared with the blood DHEA-S concentration of the subject before taking the drink, the effect from taking a specific health drink can be assessed as confirmed.

An example of the effect in the above blood LDL cholesterol concentration is a reduced blood LDL cholesterol concentration of a subject after taking the drink for a predetermined period of time.

Hereinafter, the present invention is described further in detail with reference to examples, but the technical scope of the present invention is not limited thereto. Additionally, in the experiment and the like, below, the subjects received thorough explanation on the test contents and methods before the tests started, and the tests were carried out after the written consensus was obtained.

### Example 1

[Double Blind Test]

(Test Drinks)

A double blind test was carried out using the following 3 kinds of test drinks. The general formulations of drink a, drink b, and drink c are shown in Table 1 below.

TABLE 1

| | Drink | | |
|---|---|---|---|
| | Drink a | Drink b | Drink c |
| Ingredient name | g/1000 mL | | |
| Fructose-glucose syrup | 105 | 105 | 105 |
| Erythritol | 52.5 | 52.5 | 52.5 |
| Pineapple juice | 20 | 20 | 20 |
| Honey | 10 | 10 | 10 |
| Acidifier | 6 | 6 | 6 |
| Water-soluble nucleoprotein | 5 | 5 | — |
| Oligo RNA (containing 85% or more) | 1.39 | 1.39 | — |
| Glutathione-containing yeast | — | 0.28 | 0.28 |
| (12% glutathione content) | | | |
| Edible yeast (containing zinc) | 1 | 1 | — |
| Collagen peptide (pig-derived) | 95 | 95 | — |
| Liquid dextrin | — | — | 107 |
| Mucopolysaccharide protein complex | 0.229 | 0.229 | — |
| (containing chondroitin) | | | |
| Cockscomb extract | 0.06 | 0.06 | — |
| (containing hyaluronic acid) | | | |
| Vitamin C | 12 | 12 | — |
| Sweetener (sucralose) | 0.02 | 0.02 | — |
| Water | Balance | Balance | Balance |

Drink a was prepared based on the formulation of conventional products (a nucleic acid drink: Natural DN Collagen: manufactured by FOR DAYS Co., Ltd.). Drink b is the drink of the present invention prepared by adding a high glutathione-content yeast extract to drink a as the conventional product. Table 1 shows an example of separately mixing oligo RNA and the glutathione-containing yeast, but drink b may be prepared by using MATERNAL® RNA with the equivalent composition. Drink c is a drink for studying the effect only when the ingredients considered to be the functional components and the emulsifier were removed from drink a as the conventional product and glutathione was added thereto, and a liquid dextrin was added to bring the texture of drink c closer to those of drink a and drink b. Also, when the liquid dextrin was added in place of collagen peptide, sweetness intensifies compared with drink a and drink b, and for this reason the sweetener (sucralose) was removed therefrom. Further, a flavor, preservative, and emulsifier are added to each of drinks a and b, whereas a flavor and preservative are added to c.

(Subjects)

Subjects were 60 healthy male (age 53.5±2.5, average BMI 22.3 kg/m2). A physical check-up conducted in advance confirmed that the subjects were non-smokers and have normal blood sugar concentration (glucose, HbA1C), liver functions (GOT, GPT, γ-GTP), pancreas function (amylase), uric acid concentration, kidney function (creatinine). The subjects were randomly divided into 3 groups of 20 subjects each for the subjects who take drink a (conventional product) (Group A), the subjects who take drink b (the drink of the present invention) (Group B), and the subjects who take drink c (the drink from which the active components of the conventional product are removed and a glutathione-containing yeast was added thereto) (Group C) to carry out a double blind test. The subjects were not informed of the drink they were taking but instructed to take any one of the above drinks, 20 mL each, 3 times a day at breakfast, lunch, and dinner, for 5 weeks during the test period. Additionally, the subjects were instructed to refrain from taking other supplements during the test period, but to lead the routine everyday life without practicing any particularly strict diet.

Except 1 subject from Group A and 2 subjects from Group C who stopped taking the drink in the midway, 57 subjects were tested 5 weeks after the start of taking the drink for the skin condition, POMS (Profile of Mood State), autonomic nerve measurement analysis, overall health condition screening by AMSAT, blood count, and gene analysis. The statistical procedure of the obtained data was carried out using t-test, unless otherwise specified. Also, when the assumption required for the t-test is not met, Wilcoxon's signed-rank test was used. In either case, when P value is below 0.05 (*), below 0.01 (**), below 0.001 (***), or below 0.0001 (****), a result is considered statistically significantly different.

(Investigation on Preventing or Ameliorating Effect on Skin Aging)

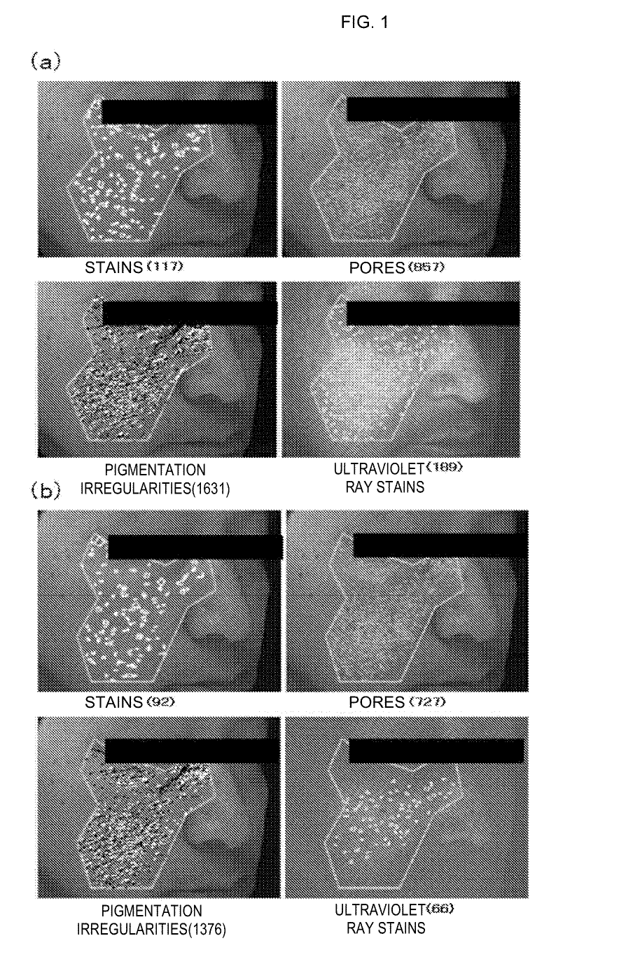

To confirm the effectiveness of the drink of the present invention to prevent or improve the skin aging, the subjects of each group were analyzed for the facial skin condition before and after taking the drink for 5 weeks, using “VISIA” which is a skin image analysis counseling system, on 6 items of stains, pores, pigmentation irregularities, ultraviolet ray stains, porphyrin, and wrinkles. Each item and the overall assessment of 6 items were analyzed by Wilcoxon's signed-rank test using statistical analysis software JMP 8. The results are shown in Table 2 below, and FIGS. 1 (a) and (b).

TABLE 2

| Measurement item by VISIA | a(n = 19) | b(n = 20) | c(n = 18) |
|---|---|---|---|
| Wrinkles | | | |
| Porphyrin | | | |
| Pores | | **↓ | |
| Ultraviolet ray stains | **↓ | ****↓ | **↓ |
| Pigmentation irregularities | *↓ | **↓ | |
| Stains | **↓ | ***↓ | *↓ |
| Degree of overall improvement | **↑ | ****↑ | *↑ |
| in 6 items | | | |

FIG. 1 (a) shows the facial skin condition of a subject before taking drink b, FIG. 1 (b) shows the facial skin condition of the same subject after taking drink b for 5 weeks. The number shown on the right side of each item is the numerical value representing stain, pore, ultraviolet ray stain, and pigmentation irregularity density, and the greater the decrease in number, the greater the improvement is. The improved condition was confirmed from the photographs of each item shown in FIGS. 1 (a) and (b). Also, as evident in Table 2, it was confirmed that the subjects of only Group B had significantly reduced pores in comparison with Group A and Group C, and also had remarkably decreased numerical values (improvement) with each item of ultraviolet ray stains, pigmentation irregularities, and stains when compared with the groups who took Group A and Group C.

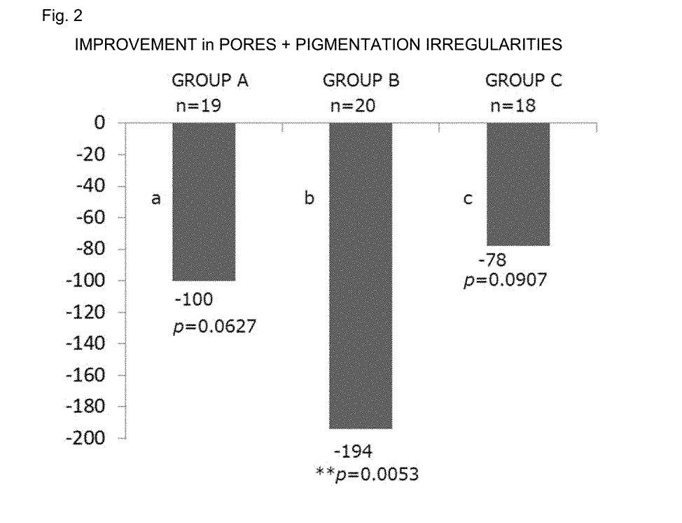

With the items of wrinkles and porphyrin, no statistically significant difference was found in any of 3 groups. However, when, with each item, the improved case was converted to 1, the case remained unchanged was converted to 0, and the aggravated case was converted to −1 to analyze the total point as the degree of overall improvement in the 6 items, it was verified that the subject group who took drink b obtained remarkable ameliorative effects (****↑). Further, only the subject group who took drink b had significantly reduced pores, but the above statistical analysis software revealed that there was a very intense correlation (p=0.0053) between the reduction (improvement) of pores and reduction (improvement) of pigmentation irregularities (see FIG. 2).

(Confirmation of the Effect by POMS)

To confirm the effectiveness of the drink of the present invention on the total mood disturbance, the subjects of each group answered POMS questionnaire, Japanese version, before and after taking the drink for 5 weeks. The results obtained using Wilcoxon's signed-rank test are shown in Table 3 below. A subject in Group B did not answer one of the items, which consequently appeared as n=19.

TABLE 3

| | | | |
|---|---|---|---|
| POMS Assessment | Group A | Group B | Group C |
| (6 items) | (n = 19) | (n = 20) | (n = 18) |

| | | |
|---|---|---|
| Assessment (T-A) | | *↓ |
| Assessment (D) | | *↓ |
| Assessment (A-H) | **(n = 19)↓ | *↓ |
| Assessment (V) | | |
| Assessment (F) | **↓ | |
| Assessment (C) | *↓ | |

In the subjects of Group B who took drink b, the items of “A-H: Anger-Hostility” (p<0.01) and F: Fatigue (p<0.01) had significantly decreased numerical values, which showed that the overall uplifting mood profile was achieved. When each question was investigated, the subjects of Group B had significantly reduced scores, when compared before and after taking the drink, on the items of “3. Angry at heart”, “7. Disappointed and discouraged”, “21. Annoyed”, “44. Dull”, “49. Uneasy”, “60. No self-esteem”, and “62. Exhausted”, whereas they had significantly increased scores on the items of “43. Can be kind-hearted to others” and “50. Full of energy”, revealing that the drink has the effects to improve or prevent stress. The ameliorative effects were not confirmed in the subjects of Group A and Group C as much of the effects confirmed in the subjects of Group B. Additionally, there was no correlation found between the elevation of DHEA-S and the improvement in the item of “A-H: Anger-Hostility” in the subjects of Group A and Group C, which is not shown in the data.

(Autonomic Nerve Function and Balance Test)

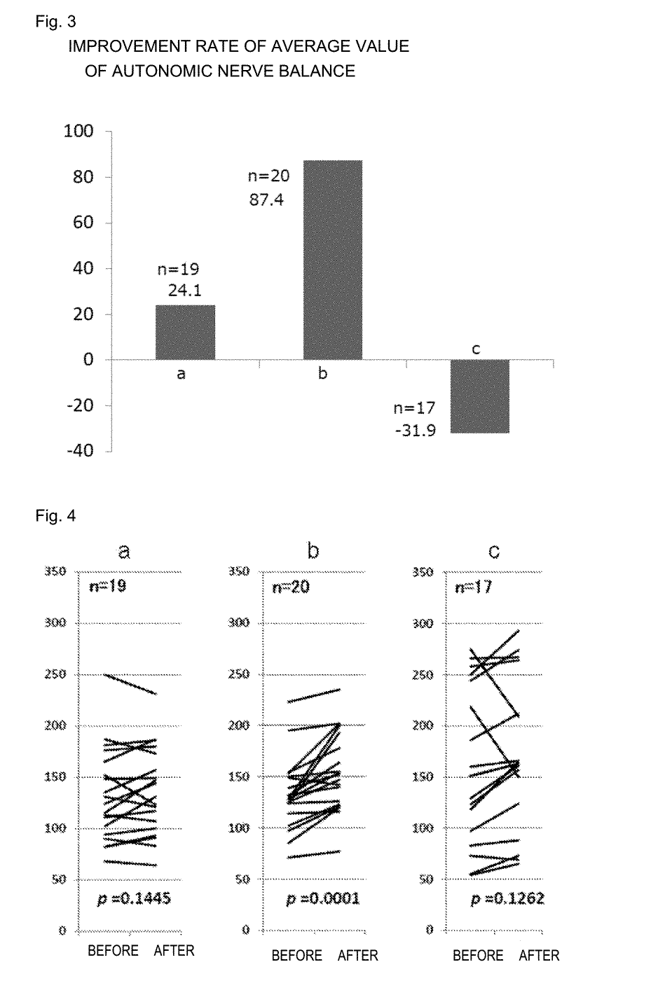

To confirm the ameliorative effects of the drink of the present invention on the autonomic nerve function and balance, the subjects of each group were assessed before and after taking the drink for 5 weeks, using “Kiritsu meijin”. In accordance with the instruction described in the manual of “Kiritsu meijin”, clips were positioned around both wrists and an electrode was attached on the chest to measure a heart rate, and a cuff was wrapped around an arm and a pulse oximeter was fixed on a finger to measure a blood pressure. Each subject kept himself at rest for 3 minutes in a sitting position, and was then instructed to stand up following the voice guidance. The measurement continued for about 3 minutes and 30 seconds after the subjects stood up. A blood pressure was measured by the oscillometric method starting 1 minute after a subject was at rest in a sitting position and 6 times per minute thereafter, and the autonomic nerve was analyzed at every beat starting after 30 seconds. The lower-sided test of Wilcoxon's signed-rank test was carried out and found that in the item of autonomic nerve balance, the subjects who took drink b had an 87.4% of improvement (see FIG. 3). Thus, it was confirmed that taking drink b provides the synergistic effects exceeding the additional effects of drink a and drink c in the autonomic nerve functions and a sense of balance, whose decline becomes problems in everyday life.

(Assessment of Subject's Overall Health Condition)

To confirm the ameliorative effects of the drink of the present invention on the subject's overall health condition, the subjects of each group were analyzed before and after taking the drink for 5 weeks, using AMSAT-HC (Auto Mastic System for Analysis Therapy—Holistic Concept). Total of 6 electrodes were attached to the forehead, both hands, both feet of each subject, and 22 patterns of electric current value were measured in 17 seconds. The measured values were compared with the data bank information stored in the system, divided to sites on the entire body, spine, and nervous system by a special algorithm including the waveform analysis, and a degree of deviation from the measurement result desired values was indicated with the maximum of ±100% at 74 sites of the entire body. Group B had more beneficial effects than Group A and Group C in the thyroid, rectum, sense organs, pharynx, thigh, hip joint, and cecum.

(Blood DHEA-S Concentration)

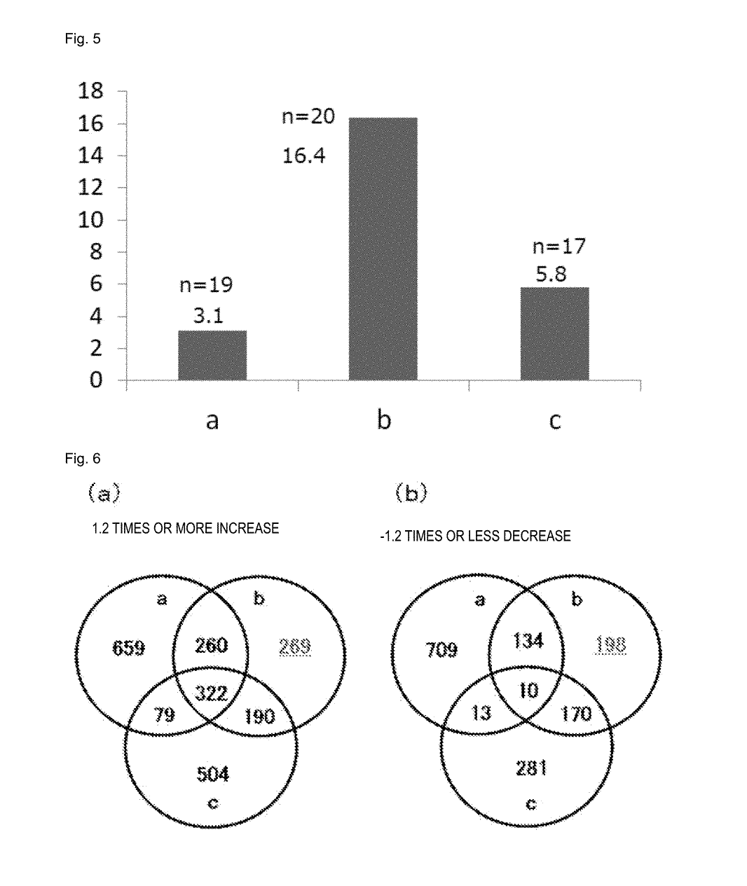

To confirm the action of the drink of the present invention on the blood components, a blood test was performed. When a blood DHEA-S concentration was measured by the RIA method, the subjects who took drink b had changes in the blood DHEA-S concentrations, which was then studied in detail. Group B, when compared with Group A and Group C, had significantly elevated blood DHEA-S concentrations with the t-test (p=0.0001) by taking drink b (see FIG. 4). Additionally, the blood DHEA-S concentration of each subject was 53 to 342 μg/dL, which was within the normal range for men in their 50s. FIG. 5 shows the average elevation rate (vertical axis) of the blood DHEA-S concentrations of the subjects in each group after as opposed to before taking each of the drinks. It was confirmed that taking drink b remarkably provides elevation of the DHEA-S concentration.

(Gene Expression Investigation)

Using blood samples collected from each subject of Group A, Group B, and Group C, expression variable genes (probe) were extracted by a statistical analysis. Using the data detected using microarray assay, the normalized expression data were subjected to the P value calculation by the significance test and a cutoff was determined based on the expression fold change. The significance test between 2 groups was carried out using SAM t-test, which is used for the 2-group test for microarray data. With the probes showing a P value of below 0.05, calculated respectively with combinations of the comparisons before and after taking the drink, the probes showing a logarithmic expression fold change between 2 groups of, in a log ratio, 0.263 or more (1.2 times or more in terms of expression fold change) were defined as a significant expression upregulating probe, whereas the probes showing a logarithmic expression fold change between 2 groups of, in a log ratio, −0.263 or less (0.8333 times or less in terms of expression fold change) were defined as a significant expression downregulating probe and used as the probe that passes a cutoff.

With each subject of Group A, Group B, and Group C, the probes showing an expression variation of 1.2 times or more increase or 1.2 times or more decrease before and after taking each of drinks a, b, and c were represented in the form of Venn diagrams for the expression increase and decrease, respectively, to indicate how the probe expression variations overlap among the groups to which the subjects belong (see FIG. 6 (a) (b)). The number of probes in which the expression was increased only in the subjects of Group B was 269 (FIG. 6 (a), underlined part), and the number of probes in which the expression was decreased only in the subjects of Group B was 198 (FIG. 6 (b), underlined part).

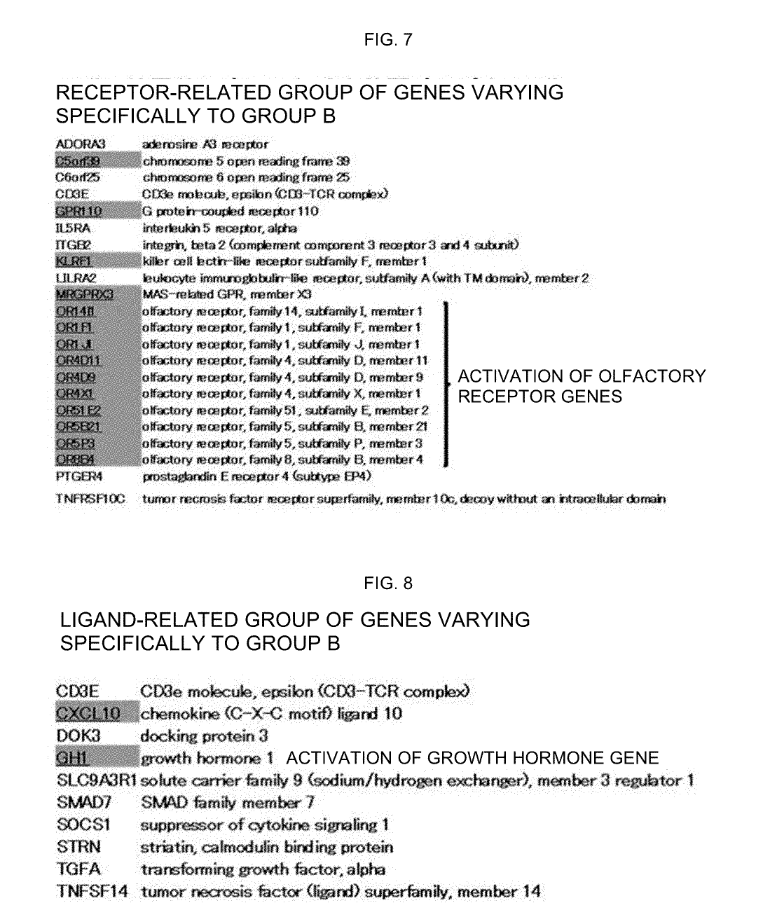

Of the probes varying specifically to the above Group B, the genes belonging to the “Receptor activity” under the molecular function of Gene Ontology were excerpted, listed, and shown in FIG. 7. The underlined gene names are the probes related to the receptors with an increased expression, whereas the gene names without underline are the probes related to the receptors with a decreased expression. Of the underlined probes, MRGPRX3, OR14I1, OR1F1, OR1J1, OR4D11, OR4D9, OR4X1, OR51E2, OR5B21, OR5P3, and OR8B4 are the genes encoding olfactory receptor proteins, and it was thus confirmed that the expression of olfactory receptor proteins is promoted. The activation of many olfactory receptor related genes is consistent with the advantageous effects found in the numerical values of the sense organ in the above assessment for overall health condition using AMSAT, thus confirming that taking the drink of the present invention renders the preventive and ameliorative effects on aging of the sense organ. On the other hand, TNF receptor TNFRSF10C and TNF ligand TNFSF14 are suppressed, but TNFRSF10C and TNFSF14 are known to have an increased expression in patients with an inflammatory disease such as arthritis, and also periodontal diseases and heart diseases (Journal of Dental Research 89 (1), 2010, 29-33, Molecular Immunology 47, 2010, 666-670, European Journal of Heart Failure 10, 2008, 352-35, etc.), thereby revealing that taking the drink or composition of the present invention suppresses the inflammatory stress.

Of the probes varying specifically to the above Group B, the genes belonging to the “Receptor binding” under the molecular function of Gene Ontology were excerpted, listed, and shown in FIG. 8. The underlined gene names are the probes related to the ligands with an increased expression, whereas the gene names without underline are the probes related to the ligands with a decreased expression. It was also confirmed that the expression of GH1 growth hormone genes was promoted. Accordingly, it was confirmed that taking the drink of the present invention is effective to prevent or improve aging.

(Conclusion)

It was confirmed that the subjects of Group B who took drink b benefit from not only the additive effects of the drink a effect and drink c effect but also the synergistic ameliorative effects in the tests on skin condition, total mood disturbance, autonomic nerve balance, and overall health condition, and also in the biological indicators such as blood DHEA-S concentration and olfactory receptor gene expression.

### Example 2

[Double Blind Crossover Trial with Identical Twin Sisters]

The trial was carried out with identical twin sisters, [FTS] (the first twin sister: FTS) and [STS] (the second twin sister: STS) (FTS BMI 34.9, STS BMI 33.6) being the subjects. [FTS] was instructed to take the drink a used in the above Example 1, 3 times a day at breakfast, lunch, and dinner, 20 mL each, for 5 weeks, a 31-day washout period, and subsequently take the drink b used in the above Example 1, 3 times a day at breakfast, lunch, and dinner, 20 mL each, for 5 weeks. [STS] was instructed to take the drink b, 3 times a day at breakfast, lunch, and dinner, 20 mL each, for 5 weeks, a 31-day washout period, and subsequently take the drink a, 3 times a day at breakfast, lunch, and dinner, 20 mL each, for 5 weeks. The total of 101 days consisting of the first 5 weeks+the 31-day washout period+the second 5 weeks is defined as the test period.

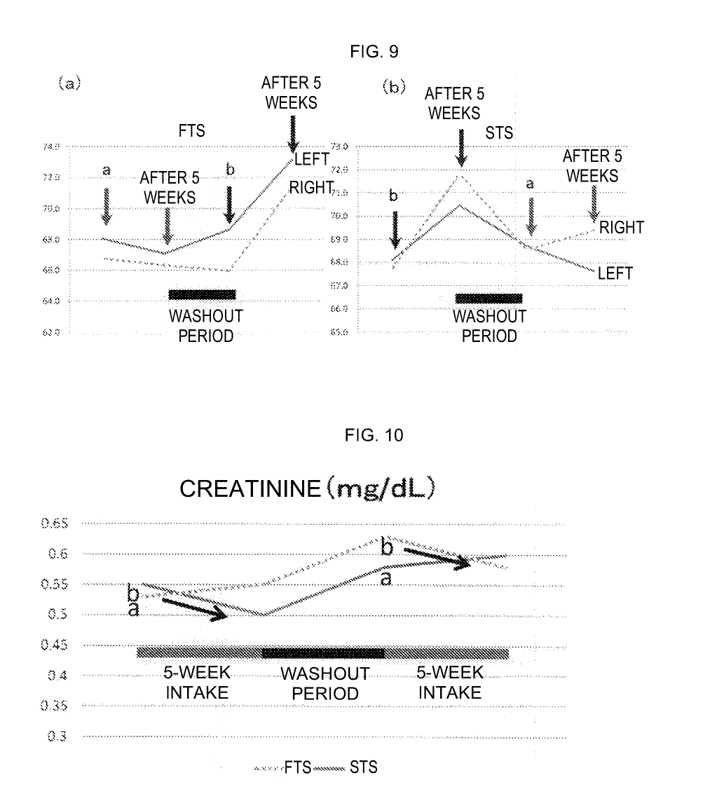

(Skin Firmness)

[FTS] and [STS] of the above identical twins were subjected to several skin firmness tests for the left and right cheeks, respectively during the test period using skin grip meter AS-GP1 (manufactured by Asahi Biomed) in accordance with the user's manual provided by the manufacture. The results are shown in FIG. 9 ((a) and (b)). [FTS] did not have increased skin firmness during the period of taking drink a and the subsequent 31-day washout period, but both left cheek (solid line) and right cheek (dotted line) had remarkably increased skin firmness during the second 5 weeks in which drink b was taken after the washout period (see FIG. 9 (a)). [STS] had increased skin firmness during the first 5 weeks in which drink b was taken, but the firmness was reduced during the 31-day washout period, and no change was substantially found during the 5 weeks in which drink a was taken (see FIG. 9 (b)).

(Creatinine Concentration)

The above [FTS] and [STS] were measured several times for the blood creatinine concentration (mg/dL) during the test period by the enzyme method (creatinase-sarcosine oxidase-peroxidase method) (measurement entrusted with Mitsubishi Chemical Medience Corporation). The results are shown in FIG. 10. The dotted line represents [FTS], and the solid line represents [STS]. [FTS] did not have a reduced blood creatinine concentration during the first 5 weeks in which drink a was taken and the subsequent 31-day washout period, but the blood creatinine concentration was remarkably reduced during the second 5 weeks in which drink b was taken after the washout period (see FIG. 10, dotted line →). [STS] had a reduced blood creatinine concentration during the first 5 weeks in which drink b was taken (see FIG. 10, solid line →), but the creatinine concentration was elevated during the 31-day washout period, and no change was found during the second 5 weeks in which drink a was taken (see FIG. 10, solid line). It is known that a creatinine concentration is elevated in patients with chronic kidney diseases or hypertension (Annals of Internal Medicine, Vol. 141 No. 12, 929-937 and Arch. Intern. Med. Vol 161, 2001, 1207-1216), thereby suggesting that the drink of the present invention is effective to improve hypertension and chronic kidney diseases.

(Total Cholesterol Concentration)

The above [FTS] and [STS] were measured several times for the blood total cholesterol concentration (mg/dL) during the test period by the enzyme method (measurement entrusted with Mitsubishi Chemical Medience Corporation). The results are shown in FIG. 11. [FTS] did not have a reduced blood total cholesterol concentration during the period in which drink a was taken and the subsequent 31-day washout period, but the total cholesterol concentration was remarkably reduced during the second 5 weeks in which drink b was taken after the washout period (see FIG. 11, dotted line →). [STS] had a reduced total cholesterol concentration during the first 5 weeks in which drink b was taken (see FIG. 11, solid line →), but the total cholesterol concentration was not elevated during the 31-day washout period, and a reducing tendency was found even during the second 5 weeks in which drink a was taken.

(LDL Cholesterol Concentration)

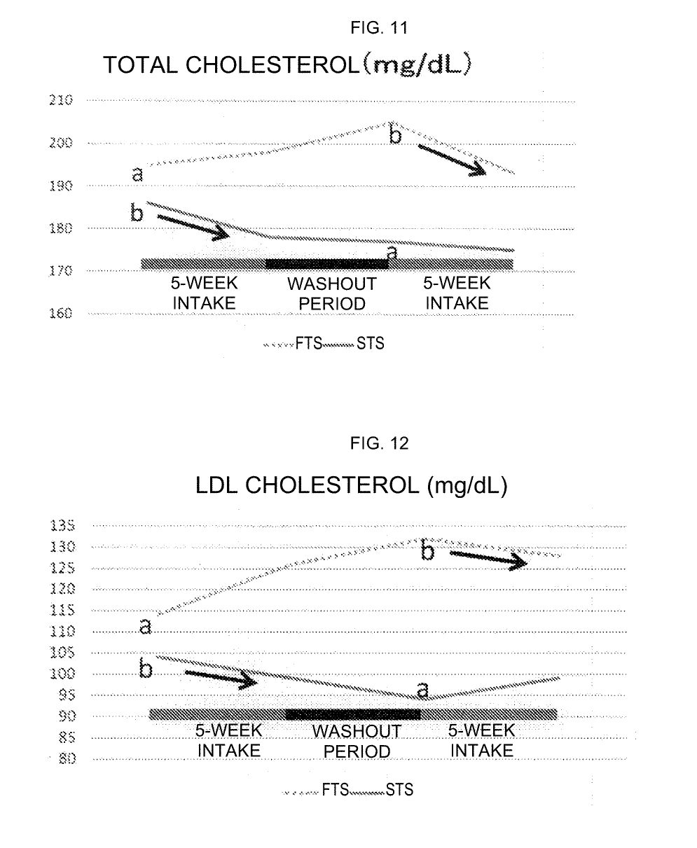

The above [FTS] and [STS] were measured several times for the blood LDL cholesterol concentration (mg/dL) during the test period by the enzyme method (measurement entrusted with Mitsubishi Chemical Medience Corporation). The results are shown in FIG. 12. [FTS] did not have a reduced blood LDL cholesterol concentration during the period in which drink a was taken and the subsequent 31-day washout period, but the LDL cholesterol concentration was reduced during the second 5 weeks in which drink b was taken after the washout period (see FIG. 12, dotted line →). [STS] had a reduced LDL cholesterol concentration during the first 5 weeks in which drink b was taken (see FIG. 12, solid line →), and the LDL cholesterol concentration was not elevated during the 31-day washout period, but a slightly increasing tendency was found during the second 5 weeks in which drink a was taken. Together with the above results of the total cholesterol concentration, it was suggested that the drink of the present invention is effective against the so-called metabolic syndrome.

(Sleep Condition)

The above [STS] was asked regarding her sleep condition, and the sleep condition problems experienced only by [STS] were improved when [STS] took drink b. Specifically, the answers such as fewer coughing at night and loud snoring were obtained. These problems were specific to STS, who is despite an identical twin, but the problems were not improved when drink a was taken.

(Investigation Using AMSAT)

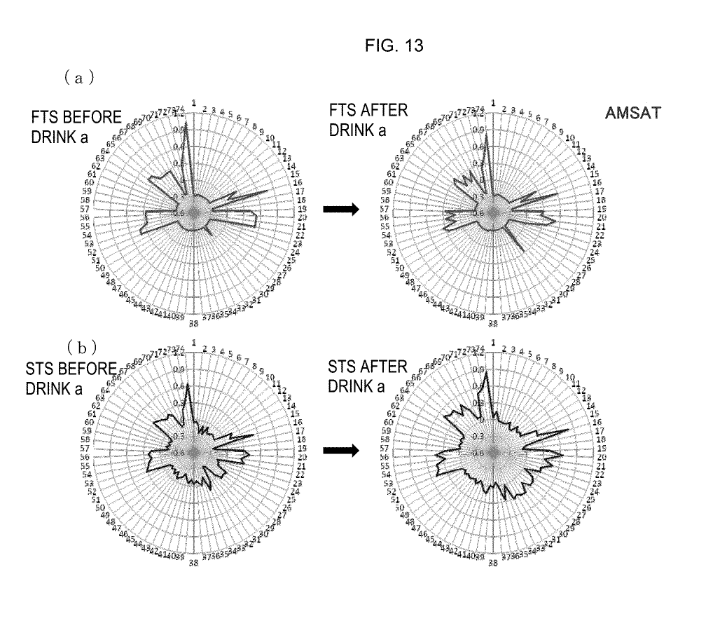

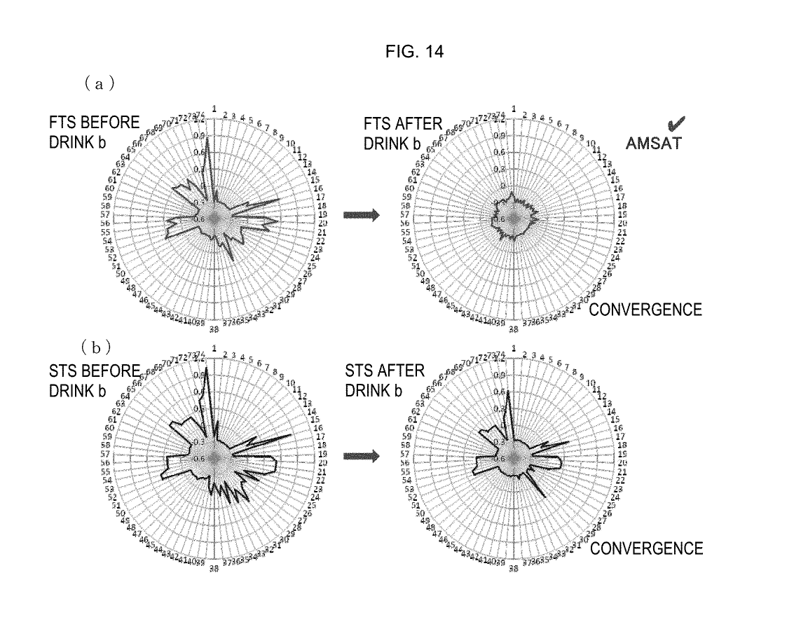

To confirm the ameliorative effects of the drink of the present invention on the subject's overall health condition, the above [FTS] and [STS] were analyzed before and after taking the drink for 5 weeks using AMSAT by the same procedure as those described in the above Example 1. When analyzed before and after taking drink a, neither [FTS] nor [STS] had remarkable change in the numerical value (see FIGS. 13 (a) and (b)), whereas when analyzed before and after the period of taking drink b, both [FTS] and [STS] had remarkably decreased numerical values, numbers close to 0, which is ideal, in most of the 74 test items after taking the drink (see FIGS. 14 (a) and (b)). Particularly, these results indicate that the softening of intercellular substances was suppressed and the ameliorative effect on inflammatory condition was found in the overall health condition assessment using AMSAT.

(Gene Expression Investigation)

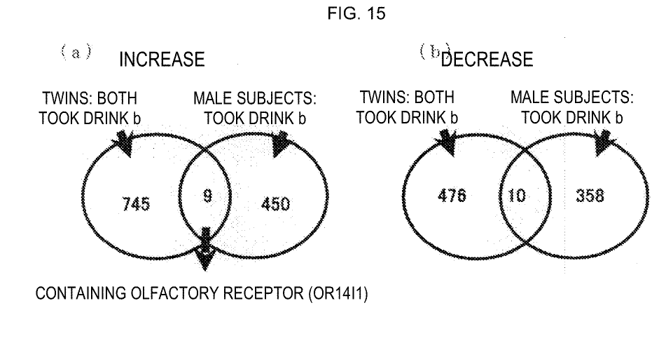

Using the blood samples from [FTS] and [STS] of the above twins who took drink b and the male subjects of Group B who took drink b in Example 1, expression variable genes (probes) were extracted by the statistical analysis with the same procedure as in the gene expression investigation of Example 1. The probes showing an expression variation of 1.2 times or more increase or 1.2 times or more decrease before and after taking drink b were represented in the form of Venn diagrams for the expression increase and decrease, respectively, to indicate how the probe expression variations overlap among the groups to which the subjects belong (see FIG. 15 (a) (b)). The number of probes in which the expression was increased was 9 in both twin subjects and male subjects. The 9 kinds of significantly increased probes (see FIG. 15 (central overlapping part)) contain the gene OR14I1 encoding olfactory receptor proteins. The activation of olfactory receptor related genes is consistent with the advantageous effects found in the numerical values of the sense organ in the above assessment for overall health condition using AMSAT.

[FTS] and [STS] of the above twins who took drink b and the male subjects of Group B who took drink b in Example 1 were subjected to the molecular function analysis of Gene Ontology. Of the genes belonging to the “metabolism activity”, the probes in which the expression was increased were subjected to the expression variation analysis at the pathway level in the pentose phosphate fundamental pathway and the TCA cycle, which appear to be particularly important. The results are shown in the following Table 4.

TABLE 4

| | | Total | | Percent- | Overlapping | | |
|---|---|---|---|---|---|---|---|
| Name | Type | Entity | Overlap | Overlap | Entities | p-Value | |
| pentose | Pathway | 35 | 5 | 14 | H6PD, PFKL, | 0.008578 | ↑ |
| phosphate | | | | | ALDOA, ALDOC, | | |
| fundamental | | | | | PFKP | | |
| pathway | | | | | | | |
| TCA cycle | Pathway | 47 | 5 | 10 | IDH3G, PCK2, | 0.028418 | ↑ |
| fundamental | | | | | CS, ACO2, SDHA | | |
| pathway | | | | | | | |

The pentose phosphate fundamental pathway is one of the glucose metabolism pathways and is an important pathway that plays a major role in metabolizing glucose and generating NADPH, which is an essential reduction power for the biosynthesis reaction of fatty acids. As evident in the above Table 4, the upregulated expression of related genes in the pentose phosphate fundamental pathway was confirmed, and it was confirmed that taking drink b works advantageously on the activation of pentose phosphate fundamental pathway.

The TCA cycle is an important cycle as the metabolism cycle for sugars, fatty acids, amino acids, and the like. As evident in the above Table 4, the upregulated expression of related genes in the TCA cycle was confirmed, and it was confirmed that taking drink b works advantageously on the energy production.

(Conclusion)

The amount of intake per kg of the body weight of drink a and drink b by the rather overweight twins are FTS 0.63 mL/kg/day and STS 0.68 mL/kg/day, which are less than the amount of intake by the male subjects in Example 1. This confirms that when drink b of the present invention is taken, even a small amount of intake, for 5 weeks, the preventive and ameliorative effects on aging and stress are provided such as the reduction of blood creatinine concentration, reduction of total cholesterol, reduction of LDL cholesterol, and improvement of sleep condition. Further, it was found that also in the expression gene analysis, taking drink b contributes to the enhancement of health condition. Taking drink b is effective to reduce the cellular stress by suppressing an inflammatory reaction caused by the physical predisposition inclined to obesity and improve the inclination to obesity by increasing basal metabolism.

1. A method of ameliorating or preventing aging or stress comprising:
identifying a subject in need of treatment for ameliorating or preventing aging or stress; and
providing the subject with an effective amount of a composition comprising 0.5-10 g of water soluble nucleoprotein, 0.1-5 g of oligo RNA, 0.1-3 g of zinc yeast, 50-150 g of collagen, 0.01-0.5 g of chondroitin, 0.01-0.5 g of hyaluronic acid, 5-20 g of vitamin C, and 6-60 mg of glutathione, each in an amount per 1000 mL, which is an amount effective to at least one of:
(i) elevate a concentration of blood dehydroepiandrosterone sulfate (DHEA-S);
(ii) promote the expression of an olfactory receptor gene; or
(iii) suppress the expression of a TNFRSF10C gene or a TNFSF14 gene,
wherein the glutathione is a yeast extract that contains more oxidized glutathione than reduced glutathione, when compared to a composition that does not comprise the glutathione.

2. The method according to claim 1, wherein the composition further comprises one or more B vitamins selected from the group consisting of vitamin B₁, vitamin B₂, vitamin B₆, and vitamin B₁₂.
